(19) United States
(12) Patent Application Publication
(10) Pub. No.: US 2018/0312907 A1
(43) Pub. Date: Nov. 1, 2018

(54) DETECTION OF BACTERIAL INFECTION

(71) Applicant: THE UNIVERSITY OF LIVERPOOL, Liverpool (GB)

(72) Inventor: Michael Griffiths, Liverpool (GB)

(21) Appl. No.: 15/770,030

(22) PCT Filed: Oct. 20, 2016
(86) PCT No.: PCT/GB2016/053269

§ 371 Date: Apr. 20, 2018

(30) Foreign Application Priority Data
Oct. 20, 2015 (GB) 1518597.8

(51) Int. Cl. C12Q 1/689

(52) U.S. Cl. CPC C12Q 1/689; C12Q 2600/158

(57) ABSTRACT

The present invention relates to methods of detecting and treating a bacterial infection, such as bacterial meningitis, and addresses the problem of misdiagnosis of bacterial infections. The medical use of antibacterial agents in the treatment of a bacterial infection in a subject identified by the methods of the invention is also described. The methods of the invention comprise assaying a sample from the subject to obtain data of the relative abundance of target molecules indicative of the expression of at least one gene pair, determining the presence or absence of a bacterial infection in the subject in dependence on the data, and optionally treating the subject with an antibacterial agent if required.

### FIELD OF THE INVENTION

The present invention relates to methods of detecting a bacterial infection, such as bacterial meningitis. The invention also relates to methods of treating a bacterial infection, such as meningitis. The invention provides the medical use of antibacterial agents in the treatment of a bacterial infection, such as bacterial meningitis, in a subject identified as having such a bacterial infection through use of a method of the invention.

### INTRODUCTION

Bacterial infection remains a significant cause of morbidity and mortality in UK and worldwide. Bacterial infection can involve body compartments, such as the brain, lungs, gut. The infection can also involve the whole body.

Bacterial infection of the brain, known clinically as meningo-encephalitis or meningitis (because of associated inflammation of the brain and/or membranes [meninges] that cover the brain), is a catastrophic infection linked with high rates of death or disability Bacterial meningitis requires emergency treatment with antibiotics and other adjunctive therapies. It is a significant cause of morbidity and mortality in the UK and worldwide. The health economic costs of bacterial meningitis are disproportionately large because many patients are left with neurological disability; the costs to society of a single case can be as high as £3-5M (Meningitis Research Foundation data).

Currently, meningitis is difficult to diagnose because, it requires a lumbar puncture (LP), to obtain a sample of cerebrospinal fluid (CSF) surrounding the brain. In many patients, the LP is not performed when needed: for some, because the patient is too critically ill, with acute seizures or coma; for others LP is delayed whilst brain scans are performed, or because of a lack of trained staff. Consequently, clinicians can start antibiotics which may later prove unnecessary, or delay starting antibiotics until all investigations are done. Delays in diagnosis or in starting anti-microbial therapy can lead to a worse patient outcome including increased mortality.

Current methods for detecting bacterial meningitis include blood cultures and lumbar puncture. Blood cultures often remain negative for a long period of time if the disease is caused by slow growing bacteria, thereby delaying an accurate diagnosis and the commencement of an optimised antibiotic. Performing a lumbar puncture on the other hand, is not suitable in all subjects, for example those who have recently taken blood thinning medication. It is also associated with an unnecessary risk and discomfort.

Additionally, subjects suspected of having bacterial meningitis are typically given empirical antibiotic treatment, despite the lack of results confirming the presence of bacterial meningitis. This often leads to unnecessary treatment with antibiotics, which increases the cost of treatment and may lead to the development of antibiotic resistance.

### SUMMARY OF THE INVENTION

In a first aspect, the invention provides a method of detecting a bacterial infection in a subject, the method comprising:
assaying a sample representative of gene expression in the subject to obtain data indicative of the relative abundance of target molecules indicative of the expression of at least at least one gene pair from the group consisting of: ELANE and IF144L; CTSG and IFI44L; ELANE and S1PR5; and CTSG and S1PR5; and
determining the presence of a bacterial infection in the subject in dependence on the data.

Suitably a method in accordance with the first aspect of the invention may comprise obtaining data indicative of the relative abundance of target molecules in respect of two or more gene pairs from those listed. For example, a method of the first aspect of the invention may comprise obtaining data indicative of the relative abundance of target molecules in respect of three or the gene pairs listed, or of all four gene pairs listed. Indeed, embodiments of the first aspect of the invention that obtain data indicative of the relative abundance of target molecules in respect of the four gene pairs: ELANE and IF144L; CTSG and IFI44L; ELANE and S1PR5; and CTSG and S1PR5; and determine the presence of a bacterial infection in the subject in dependence on such data represent particularly useful methods of the invention.

In a second aspect, the invention provides a method of detecting a bacterial infection in a subject, the method comprising:
assaying a sample representative of gene expression in the subject to obtain data indicative of the relative abundance of target molecules indicative of the expression of at least at least one gene pair from the group consisting of: ELANE and IFI44L; CTSG and IFI44L; ELANE and S1PR5; CTSG and S1PR5; GRK6 and TXLNA; DUSP1 and IFI44L; APMAP and KMT2A; DUSP1 and HP1BP3; FLOT1 and NMT1; APMAP and HUWE1; ELANE and SIGLEC1; ELANE and IFI27; and DUSP1 and NMT1; and
determining the presence of a bacterial infection in the subject in dependence on the data.

A method of detecting a bacterial infection in accordance with the invention, in which a bacterial infection is detected in a subject, may further comprise a step of conducting a further confirmatory test for the bacterial infection in the subject. Suitably, in such an embodiment, a bacterial infection is diagnosed when the results of both the first test (a method of the invention) and the second test (the confirmatory test) indicate the presence of bacterial infection. For example, a sample of biological fluid, such as CSF from lumbar puncture or blood from venepuncture, may be cultured in order to grow the microorganism associated with infection. This allows confirmation of bacterial infection and also allows determination of antimicrobial susceptibility of any isolated pathogen.

A method of the first or second aspect of the invention, in which a bacterial infection is detected in a subject, may further comprise a step of providing the subject with treatment for the bacterial infection.

Indeed, in a third aspect, the invention provides a method of treating a bacterial infection in a subject, the method comprising:
assaying a sample representative of gene expression in the subject to obtain data indicative of the relative abundance of target molecules indicative of the expression of at least at least one gene pair from the group consisting of: ELANE and IF144L; CTSG and IFI44L; ELANE and S1PR5; and CTSG and S1PR5;
determining the presence of a bacterial infection in the subject in dependence on the data; and
   providing treatment for a bacterial infection to the subject when the determination indicates the presence of a bacterial infection in the subject.

Indeed, in a fourth aspect, the invention provides a method of treating a bacterial infection in a subject, the method comprising:
assaying a sample representative of gene expression in the subject to obtain data indicative of the relative abundance of target molecules indicative of the expression of at least at least one gene pair from the group consisting of: ELANE and IFI44L; CTSG and IFI44L; ELANE and S1PR5; CTSG and S1PR5; GRK6 and TXLNA; DUSP1 and IFI44L; APMAP and KMT2A; DUSP1 and HP1BP3; FLOT1 and NMT1; APMAP and HUWE1; ELANE and SIGLEC1; ELANE and IFI27; and DUSP1 and NMT1;
determining the presence of a bacterial infection in the subject in dependence on the data; and
   providing treatment for a bacterial infection to the subject when the determination indicates the presence of a bacterial infection in the subject.

The treatment for a bacterial infection, such as bacterial meningitis, provided in a method of the third or fourth aspects of the invention may comprise providing the subject requiring such treatment with a therapeutically effective amount of an antibacterial agent.

In a fifth aspect the invention provides an antibacterial agent for use in the treatment of a bacterial infection in a subject identified as having a bacterial infection by a method in accordance with either the first or second aspects of the invention.

Methods in accordance with the first, second, third, or fourth aspects of the invention may comprise obtaining data indicative of the relative abundance of target molecules indicative of the expression further gene pairs, as long as this is in addition to obtaining data indicative of relative abundance of at least one of the gene pairs referred to above.

Suitable examples of further gene pairs, and the benefits that may be gained by employing such further pairs, are described elsewhere in the present disclosure.

Merely by way of example, further gene pairs particularly suitable for use in embodiments of the methods of the first or third aspects of the invention may include at least one of: GRK6 and TXLNA; DUSP1 and IFI44L; C20ORF3 and MLL; DUSP1 and HP1BP3; ELANE and SIGLEC1; or ELANE and IFI27.

In a sixth aspect, the invention provides a method of detecting the relative abundance of target molecules in a subject suspected of having or developing a bacterial infection, the method comprising:
Providing a sample representative of gene expression in the subject suspected of having or developing a bacterial infection;
Detecting the relative abundance of target molecules indicative of the expression of at least one gene pair, wherein the gene pair is selected from the group according to any of the first, second, third or fourth aspects of the invention.

Except for where the context requires otherwise, references to the methods of the invention may be taken as encompassing the detecting methods of the first, second or sixth aspects of the invention, or the methods of treatment of the third or fourth aspects of the invention. Embodiments disclosed in respect of one method of the invention should also be taken as applicable to the other methods of the invention, unless incompatible, or otherwise stated.

### DETAILED DESCRIPTION OF THE INVENTION

The methods of the present invention provide simple tests that may be used in the diagnosis of bacterial infections. Such infections include, but are not limited to, bacterial meningitis. The advantages provided in respect of bacterial meningitis are notable, and worthy of further comment at this time.

As discussed elsewhere in the specification, the methods of the invention may be put into practice in the form of a blood test. The use of a blood sample to detect bacterial infections of the brain, such as bacterial meningitis, provides advantages in that it avoids the need for the more invasive lumbar punctures generally used in accepted prior art techniques for the diagnosis of bacterial meningitis.

As describe herein, a sample of biological fluid, such as CSF from lumbar puncture or blood from venepuncture, may be cultured in order to grow the microorganism associated with infection. This allows confirmation of bacterial infection and also allows determination of antimicrobial susceptibility of any isolated pathogen. The disadvantage of culture is that it typically takes 48 hours or longer to grow a microorganism. In contrast the methods of invention (for example when used on a PCR platform), are able to provide an indication of bacterial infection 2-3 hours from sample receipt, allowing prompt initiation of antibiotics. Since the methods of the invention allow the prompt initiation of antibiotics, the additional combination of optional confirmatory steps need not unduly delay treatment.

Methods of the invention, such as those employing a blood test, may allow patients with suspected bacterial meningitis to be identified swiftly, and guide medical staff to commence appropriate treatment promptly. Furthermore, such methods allow patients without meningitis to avoid unnecessary antibiotics and be discharged from hospital quicker.

Bacterial meningitis patients can present with a range of clinical symptoms and signs, such as headache, neck-stiffness, and dislike of bright lights (photophobia). However, some or all of these symptoms can be absent in individual subjects. As demonstrated in the Experimental Results, the methods of the invention are able to distinguish proven bacterial meningitis from ‘look-a-like’ conditions, such as meningism or viral meningitis that may give rise to symptoms that may otherwise be mistaken for those of bacterial meningitis.

Currently, bacterial meningitis is diagnosed by; (i) an elevated number of white cells in the CSF and (ii) detection of bacteria (by bacterial culture or a bacteria specific nucleic acid amplification test [NAAT]). Patients with meningism often have clinically indistinguishable symptoms and signs to bacterial meningitis, but lumbar punctures conducted on such subjects demonstrate that there are none or very few white cells in the CSF (<4 cells/μl). Patients with viral meningitis, again can have clinically indistinguishable symptoms and signs to bacterial meningitis, but these cases lumbar punctures detect virus in the CSF (via viral specific NAAT).

The methods of the invention allow the detection of bacterial infections, such as bacterial meningitis, even in cases where the sample is collected after the subject has received antibiotics and/or other anti-microbial treatment. This is in contrast to conventional methods for detecting bacteria, whether by culture or by NAAT, which often show poor sensitivity (fail to detect true cases of bacterial infection) when patient samples are collected after anti-microbial treatment has been initiated.

The combinations of host biomarker gene pairs used in the methods of the invention have been deliberately selected using patient blood samples collected both before and after (up to 6 weeks) initiation of anti-microbial treatment. Thus the invention offers to reduce the risk of missing true cases of bacterial meningitis among patients who have previously begun treatment with antibiotics.

By measuring human biomarkers in blood, this invention offers to revolutionise, accelerate and improve diagnosis of bacterial infections, guide prompt provision of appropriate antibiotic management and enhance patient outcomes. This is of particular benefit in the case of bacterial infections of the CNS, such as bacterial meningitis.

In order to assist the understanding of the present invention, certain terms used herein will now be further defined in the following paragraphs.

Bacterial Infection

A “bacterial infection”, for the purposes of the present disclosure, may be taken as referring to any undesired presence and/or growth of bacteria in a subject. Such undesired presence of bacteria may have a negative effect on the host subject's health and well-being.

While the term “bacterial infections” should not be taken as encompassing the growth and/or presence of bacteria which are normally present in the subject, for example in the digestive tract of the subject, it may encompass the pathological overgrowth of such bacteria.

Bacterial infections may be caused by the growth and/or presence of gram positive and/or gram negative bacteria.

Bacterial infections may be caused by a wide range of bacteria. Merely by way of example, bacteria which are typically associated with meningo-encephalitis in human subjects may include: Streptococcus pneumoniae, Neisseria Meningitides. Haemophilus influenzae, Streptococcus agalactiae, Listeria monocytogenes, Escherichia coli and Mycobacterium tuberculosis.

Other bacteria involved in human infection of the central nervous system, particularly when subjects have indwelling catheters include: Staphylococcus aureus, Pseudomonas aeruginosa and Ureaplasma urealyticum.

Bacteria involved in human infection outside the central nervous system include: Moraxella catarrhalis, Clostridium perfringens, Neisseria gonorrheae, Chlamydia trachomatis, Helicobacter pylori, Campylobacter jejuni, Salmonella enterica, Enterococcus faecalis, Clostridum difficile, Staphylococcus saprophyticus, Treponema pallidum, Haemophilus ducreyi, Mycoplasma pneumoniae, Chlamydia pneumoniae, and Legionella pneumophila.

The inventors believe that the methods or medical uses of the invention are applicable to bacterial infections in which the bacterial pathogens are selected from any of the groups set out above.

The methods or medical uses of the present invention are applicable to bacterial infection in any system, organ or area of the subject, such as but not limited to, gastrointestinal system, respiratory system, urinary system, the ocular system, the auditory system and the skin.

The inventors' results have also demonstrated that bacterial infections such as sepsis (blood stream infection) and pneumonia (respiratory bacterial infection) may be detected using the methods of the invention.

The methods and medical uses of the invention are believed to have utility in helping to diagnose bacterial infections of prosthetic implants. These may include implants in the head or other body compartments. Merely by way of suitable example of a prosthetic head implant, the Experimental Results demonstrate that methods of the invention are able to correctly distinguish bacterial infection from mechanical failure in ventriculo-peritoneal (VP) shunts, using either blood or CSF samples. It is anticipated the methods of the invention would work equally well using body fluid from other implants, such as CSF from external ventricular drains in the head, or synovial fluid from the joint space around prosthetic knee replacements.

Without detracting from the above, the methods and uses of the invention are particularly suitable for use in connection with bacterial infections in the central nervous system, such as bacterial meningitis.

Bacterial Meningitis

The term “bacterial meningitis”, as used herein, refers to inflammation of the meninges caused by any form of bacteria. Merely by way of example, bacterial meningitis may be caused by bacteria selected from the group consisting of: Streptococcus pneumoniae, Neisseria Meningitides, Haemophilus influenzae, Streptococcus agalactiae, Listeria monocytogenes and Escherichia coli.

As illustrated by the results presented in the Experimental Results section, the methods of the invention are able to distinguish between bacterial meningitis and other disorders of the meninges, including meningism and viral meningitis. References in the present specification to detection or diagnosis of bacterial meningitis should be construed as allowing such distinctions to be made, in keeping with the specificity and selectivity of the methods discussed further herein.

Detecting Bacterial Infections

The methods of the first, second and sixth aspects of the invention are methods for detecting bacterial infections in a subject. In the context of the present disclosure, the term “detecting” is taken as meaning determining the presence of a bacterial infection in a subject. Suitably, and as discussed further below, the methods of the invention may be used to detect bacterial meningitis.

In one embodiment, the method of the invention may be used to detect a bacterial infection that is associated with symptoms in a subject. Alternatively the methods may also be used to detect a bacterial infection that is essentially asymptomatic. Such asymptomatic bacterial infections may be infections that are at a very early stage of the disease.

The inventors believe that the assessment, in the methods of the invention, of expression of genes associated with the subject's host response to bacterial infections to provide information regarding the presence of this condition confers notable advantages. Without wishing to be bound by any hypothesis, one such advantage may be that target molecules indicative of expression of such genes are found at detectable levels at earlier time points than are potential markers expressed by the bacteria associated with the infection. While levels of expression of bacterial genes may be difficult to detect, especially during the early stages of bacterial infections, the expression of host genes may be readily assessed. The greater abundance of such target molecules indicative of expression of host response genes not only allows such assessments to be made at an earlier time, but may also improve the accuracy of the assessment.

Detecting Bacterial Meningitis

The methods of the invention are methods are particularly suitable for use in detecting bacterial meningitis in a subject.

Once bacterial meningitis has been detected by a method of the invention an additional step may optionally be practiced, to confirm the detection. The additional step may comprise performing a further diagnostic test for bacterial meningitis in respect of the subject. Such a further diagnostic test may be used to identify the bacterial pathogen present, or to provide information regarding the susceptibility of the bacterial pathogen to antimicrobial agents.

Examples of such diagnostic tests that may be employed will be well known to those skilled in the art. Suitably the further diagnostic test may comprise a lumbar puncture. Bacteria collected as part of the further diagnostic test may be investigated by culturing or by NAAT as described elsewhere in the specification.

It will be appreciated that, even when a lumbar puncture is used to confirm the detection or diagnosis provided by a method of the invention, the use of the methods of the invention significantly reduces the number of incidences of lumbar puncture that are needed (since lumbar puncture may be avoided in those cases where the methods of the invention indicate that bacterial meningitis is not present).

A Subject

The methods and medical uses are practiced in respect of a subject. The subject may be one requiring diagnosis or treatment for a bacterial infection, such as bacterial meningitis. Suitably the subject may be a human subject. The subject may be a patient undergoing medical care, or an individual requesting medical care.

In the methods of the first, second or sixth aspect of the invention, a suitable subject may be one believed to have a bacterial infection, such as bacterial meningitis. For example, a suitable subject may have symptoms consistent with a bacterial infection, such as bacterial meningitis. In such cases the methods of the invention may be of use in definitively detecting or diagnosing the presence of a bacterial infection, such as bacterial meningitis. Alternatively a suitable subject may lack some or all symptoms consistent with a bacterial infection, such as bacterial meningitis. As considered elsewhere in this specification, such a subject may be substantially asymptomatic.

Alternatively, a suitable subject in the context of the first, second or sixth embodiments of the invention may be one believed to be at risk of developing a bacterial infection, such as bacterial meningitis. Such a subject may have been in contact with an individual suffering from a bacterial infection and consequently may be believed to be at elevated risk of developing a bacterial infection as a result of this contact.

The ability of the methods of the invention to detect or diagnose a bacterial infection, such as bacterial meningitis, in a timely manner, and without the need for invasive procedures such as lumbar puncture, may be of benefit to any of the groups of subjects identified above.

It will be appreciated that a subject who may gain benefit from the methods of treatment of the third or fourth aspects of the invention is one in whom a bacterial infection is determined to be present by the assessment and comparisons conducted as part of the methods of the first, second or sixth aspects of the invention.

A Sample Representative of Gene Expression in a Subject

The methods of the invention make use of samples that are representative of gene expression in the subject in respect of whom the method is being practiced. In particular, the methods of the invention use samples representative of expression of host response genes involved in a subject's response to bacterial infection. Methods of the invention may make use of any sample which contains target molecules that provide a representation of such gene expression in the subject in question.

In suitable embodiment, the sample is a body fluid sample. Alternatively, suitable samples may include tissue samples, such as biopsies. In either case, a suitable sample (whether of a body fluid or tissue) may comprise biological cells from the subject that are involved in the host response to bacterial infection.

A suitable body fluid sample may include: a blood sample (for example, a whole blood sample, a blood plasma sample, or a serum sample); or a cerebrospinal fluid (CSF) sample, or a synovial fluid sample.

Blood samples are particularly suitable for use in the methods of the invention. The use of a blood sample (whether whole blood, plasma, or serum) is advantageous, since it is readily accessible. Additionally obtaining the sample is not associated with much less risk and patient discomfort than is the case for samples such as CSF.

Samples may be processed for the enrichment of target molecules. Suitable techniques for such enrichment may be determined with reference to the nature of the sample, and of the target molecule in question. Generally, examples of suitable techniques (such as techniques for the isolation of biological cells from a sample, and the preparation of the cells to yield gene transcripts) will be well known to those skilled in the art.

Target Molecules

Target molecules suitable for use in the method of the invention are any molecules which are representative of gene expression in the subject. Such target molecules may be representative of gene expression either directly or indirectly. By way of example, a suitable target molecule which is directly representative of gene expression may comprise an RNA transcript. A suitable target molecule which is indirectly representative of gene expression may comprise a protein encoded by the gene.

It will be appreciated that the selection of the type of target molecule to be used in a method of the invention should be considered in connection with the nature of the sample representative of gene expression. In the case of a sample that contains host cells, a suitable target molecule may be either directly indicative of gene expression (such as an RNA transcript), or indirectly indicative of gene expression (such as a protein encoded by an expressed gene). In the case of a sample that is essentially acellular, the use of target molecules that are indirectly indicative of gene expression may be preferred, since such target molecules (an in particular protein examples of such target molecules) may be shed into the sample, even in the absence of biological cells.

By the same token, the nature of the target molecule, and therefore the nature of the sample representative of gene expression, may be chosen in order to be consistent with use in a preferred assaying system.

Assaying

When practicing the method of the invention, the sample is assayed to determine the relative abundance of the target molecules of interest. Generally, techniques suitable for assaying gene expression in order to provide information regarding relative abundance will be known to the person skilled in the art. As set out above, a suitable assay technique may be chosen with reference to the nature of the sample and the target molecules.

Merely by way of example, gene expression can be measured directly by techniques that allow the detection and quantification of RNA target molecules, such as RT-PCR, real-time PCR, Northern blot, RNA sequencing (RNA-seq) and RNA microarray.

Gene expression can be measured indirectly, by techniques that allow the detection and quantification of protein target molecules, such as ELISA, radioimmunoassay, immunoprecipitation, Western blot and mass spectrometry. Other suitable techniques for assaying proteins may be known to the person skilled in the art.

In a suitable embodiment an assay may allow the relative abundance of multiple sets of target molecules to be determined within a single reaction. An assay meeting such requirements may be referred to as a multiplex assay. Suitably a multiplex assay may allow the relative abundance of all requisite target molecules to be determined within a single reaction mixture (a “single tube” multiplex assay). Single tube multiplex assays may be particularly suitable for determining the relative abundance of mRNA transcript target molecules within a sample.

Data Indicative of Relative Abundance of Target Molecules

The methods of the invention involve obtaining data indicative of the relative abundance of relevant target molecules. For the purposes of the present invention, such “data indicative of relative abundance” may be taken as encompassing any data that are capable of being used in determining the presence of a bacterial infection, such as bacterial meningitis, in a subject. In a suitable embodiment the data provide a quantitative indication of relative abundance of expression of members of gene pairs, for example with reference to relevant target molecules.

Genes and Gene Pairs Suitable for Use in the Methods of the Invention

The methods of the invention involve obtaining data indicative of the relative abundance of target molecules indicative of the expression of at least one of the gene pairs referred to in the first or second aspects of the invention. These gene pairs are made up of first and second host response genes.

The list of genes from which the gene pairs referred to herein are composed are set out in Table 1, which also provided details of the products encoded by these genes. Particular groups of gene pairs of interest that may be usefully employed in the methods of the are set out in Tables 2 to 5.

The expression of the genes and gene pairs is investigated by assaying the sample for target molecules indicative of the expression of such host response genes, as referred to above.

Table 2

The methods of the first aspect of the invention involve obtaining data indicative of the relative abundance of target molecules indicative of the expression of at least at least one gene pair from the group consisting of: ELANE and IF144L; CTSG and IFI44L; ELANE and S1PR5; and CTSG and S1PR5. This group of gene pairs is set out in Table 2.

In a suitable embodiment a method of the first aspect of the invention may involve obtaining data indicative of the relative abundance of target molecules indicative of the expression of at least two, or at least three of the gene pairs set out in Table 2, and determining the presence of a bacterial infection in the subject in dependence on the data in respect of each pair for which data is obtained. In a suitable embodiment, a method in accordance with the first aspect of the invention may involve obtaining data indicative of the relative abundance of target molecules indicative of the expression of all four of the gene pairs set out in Table 2, and determining the presence of a bacterial infection in the subject in dependence on the data in respect of all four of these pairs.

Table 3

In a suitable embodiment, a method of the second aspect of the invention involves obtaining data indicative of the relative abundance of target molecules indicative of the expression of one, two, three, four, five, six, seven, eight, nine, ten, eleven, twelve or thirteen pairs of genes selected from the group consisting of: ELANE and IFI44L; CTSG and IFI44L; ELANE and S1PR5; CTSG and S1PR5; GRK6 and TXLNA; DUSP1 and IFI44L; APMAP and KMT2A; DUSP1 and HP1BP3; FLOT1 and NMT1, APMAP and HUWE1, ELANE and SIGLEC1; ELANE and IFI27; and DUSP1 and NMT1. This group of gene pairs is set out in Table 3.

Suitably, a method of the invention may involve obtaining data indicative of the relative abundance of target molecules indicative of the expression of at least one, at least two, at least three, at least four, at least five, at least six, at least seven, at least eight, at least nine, at least ten, at least eleven, or at least twelve of the gene pairs set out in Table 3, and determining the presence of a bacterial infection in the subject in dependence on the data in respect of each pair for which data is obtained.

In a suitable embodiment a method of the invention involves obtaining data indicative of the relative abundance of target molecules indicative of the expression of two, up to three, up to four, up to five, up to six, up to seven, up to eight, up to nine, up to ten, up to eleven, or up to twelve of the gene pairs set out in Table 3, and determining the presence of a bacterial infection in the subject in dependence on the data in respect of each pair for which data is obtained.

In a suitable embodiment a method of the invention may involve obtaining data indicative of the relative abundance of target molecules indicative of the expression of all thirteen of the gene pairs set out in Table 3, and determining the presence of a bacterial infection in the subject in dependence on the data in respect of all thirteen of these gene pairs.

Table 4

In a further embodiment, the method of the invention involves obtaining data indicative of the relative abundance of target molecules indicative of the expression of one, two, three, four, five or six pairs of genes selected from the group consisting of: GRK6 and TXLNA; DUSP1 and IFI44L; APMAP and KMT2A; DUSP1 and HP1BP3; APMAP and HUWE1; and DUSP1 and NMT1. This group of gene pairs is set out in Table 4.

Suitably, a method of the invention may involve obtaining data indicative of the relative abundance of target molecules indicative of the expression of at least one, at least two, at least three, at least four, or at least five of the gene pairs set out in Table 4, and determining the presence of a bacterial infection in the subject in dependence on the data in respect of each pair for which data is obtained.

In a suitable embodiment a method of the invention involves obtaining data indicative of the relative abundance of target molecules indicative of the expression of two, up to three, up to four, or up to five of the gene pairs set out in Table 4, and determining the presence of a bacterial infection in the subject in dependence on the data in respect of each pair for which data is obtained.

In a suitable embodiment a method of the invention may involve obtaining data indicative of the relative abundance of target molecules indicative of the expression of all six of the gene pairs set out in Table 4, and determining the presence of a bacterial infection in the subject in dependence on the data in respect of all six of these gene pairs.

Table 5

In a suitable embodiment, the method of the invention involves obtaining data indicative of the relative abundance of target molecules indicative of the expression of one, two, three, four, five, six, seven, eight, nine, or ten pairs of genes selected from the group consisting of: ELANE and IFI44L; CTSG and IFI44L; ELANE and S1PR5; CTSG and S1PR5; GRK6 and TXLNA; DUSP1 and IFI44L; APMAP and KMT2A; DUSP1 and HP1BP3, ELANE and SIGLEC1; and ELANE and IFI27. This group of gene pairs is set out in Table 5.

Suitably, a method of the invention may involve obtaining data indicative of the relative abundance of target molecules indicative of the expression of at least one, at least two, at least three, at least four, at least five, at least six, at least seven, at least eight, or at least nine of the gene pairs set out in Table 5, and determining the presence of a bacterial infection in the subject in dependence on the data in respect of each pair for which data is obtained.

In a suitable embodiment a method of the invention involves obtaining data indicative of the relative abundance of target molecules indicative of the expression of two, up to three, up to four, up to five, up to six, up to seven, up to eight, or up to nine of the gene pairs set out in Table 5, and determining the presence of a bacterial infection in the subject in dependence on the data in respect of each pair for which data is obtained.

In a suitable embodiment a method of the invention may involve obtaining data indicative of the relative abundance of target molecules indicative of the expression of all ten of the gene pairs set out in Table 5, and determining the presence of a bacterial infection in the subject in dependence on the data in respect of all ten of these gene pairs.

In a suitable embodiment, a method of the invention may be based upon the use of data only in respect of genes, or gene pairs, set out in the preceding paragraphs. For example, a method of the invention may make use only of data in respect of the set of gene pairs set out in Table 2. Suitably, a method of the invention may make use only of data in respect of the set of gene pairs set out in Table 3. In a suitable embodiment a method of the invention may make use only of data in respect of the set of gene pairs set out in Table 4. Alternatively a method of the invention may make use only of data in respect of the set of gene pairs set out in Table 5.

In another suitable embodiment, the methods of the invention may employ one or more additional gene pairs beyond those set out in Table 3.

Determining the Presence of a Bacterial Infection

Suitably, in a method of the invention, the step of determining comprises using the data to calculate one or more functions, where the presence of a bacterial infection in the subject is determined to be present if one or more of the calculated functions satisfies a predetermined condition.

In a suitable embodiment, the one or more functions includes a logical function. In another suitable embodiment, the one or more functions includes a linear function. Examples of such embodiments are set out in the Experimental Results below.

Suitably the function is a linear discriminant function. An example of a suitable linear discriminant function that may be employed in the methods of the invention is described in the Experimental Results section.

Suitably the predetermined condition is satisfied if the calculated linear discriminant function exceeds a predetermined threshold. An example of a suitable predetermined threshold that may be employed with reference to a linear discriminant function is described in the Experimental Results section.

Treatment of Bacterial Infections, Such as Bacterial Meningitis

The present invention consider the treatment of bacterial infections, such as bacterial meningitis not only in the methods of treatment and medical uses described herein, but also in certain embodiments of the methods of detecting or diagnosing bacterial meningitis.

Treatment of bacterial infections, such as bacterial meningitis, in the context of the present disclosure will generally involve the provision of antibacterial agents to a subject requiring such treatment. Suitable antibacterial agents include antibiotics such as those selected from the group consisting of: Third generation cephalosporins (e.g. ceftriaxone, cefotaxime), ampicillin, penicillin G, meropenem, fluoroquinolone, trimethoprim-sulfamethoxazole, and chloamphenicol.

Suitably an antibacterial agent for use in accordance with this aspect of the invention may be selected with reference to the nature of the bacteria giving rise to the bacterial infection, such as bacterial meningitis, to be treated.

Thus, in the case of a bacterial infection with Streptococcus pneumoniae an antibacterial agent for use in accordance with this aspect of the invention may be selected from the group of: vancomycin plus a third-generation cephalosporin; ceftriaxone or cefotaxime; Meropenem (C-III), fluoroquinolonec (B-II).

Thus, in the case of a bacterial infection with Neisseria meningitidis an antibacterial agent for use in accordance with this aspect of the invention may be selected from the group of: a third-generation cephalosporin; Ceftriaxone; Penicillin G, ampicillin, chloramphenicol, fluoroquinolone, aztreonam.

Thus, in the case of a bacterial infection with Listeria monocytogenes an antibacterial agent for use in accordance with this aspect of the invention may be selected from the group of: Ampicillin (optionally with addition of an aminoglycoside) or penicillin G (optionally with addition of an aminoglycoside) Trimethoprim-sulfamethoxazole, meropenem (B-III).

Thus, in the case of a bacterial infection with Streptococcus agalactiae an antibacterial agent for use in accordance with this aspect of the invention may be selected from the group of: Ampicillind or penicillin G (optionally with addition of an aminoglycoside) Third-generation cephalosporins (B-III).

Thus, in the case of a bacterial infection with Haemophilus influenzae an antibacterial agent for use in accordance with this aspect of the invention may be selected from the group of: Third-generation cephalosporin, Ceftriaxone; (A-I) Chloramphenicol, cefepime (A-I), meropenem (A-I), fluoroquinolone.

Thus, in the case of a bacterial infection with Escherichia coli an antibacterial agent for use in accordance with this aspect of the invention may be selected from the group of: Third-generation cephalosporin, ceftriaxone, (A-II) Cefepime, meropenem, aztreonam, fluoroquinolone, trimethoprim-sulfamethoxazole.

The invention will now be further described with reference to the accompanying Experimental Results and Figures, in which:

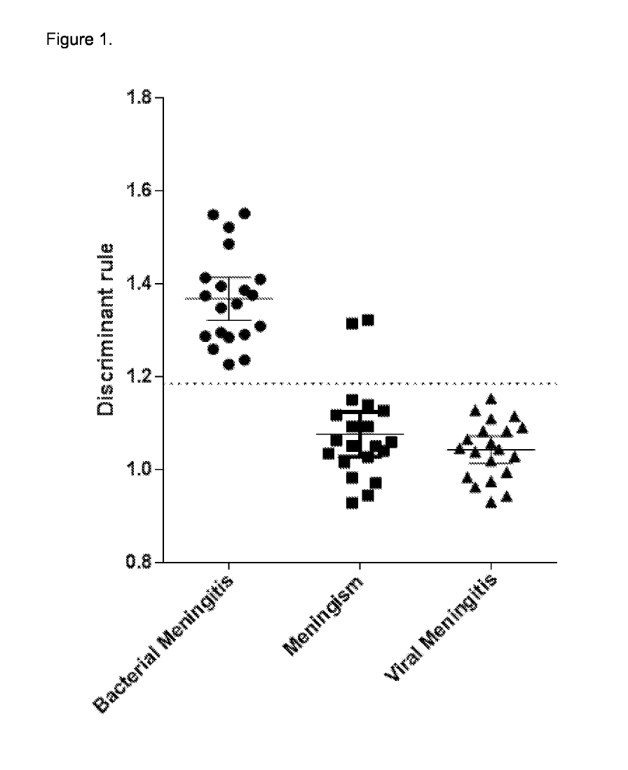

FIG. 1 is a scatter plot of overall relative abundance ratio (formulated via discriminant rule) for 4 transcript host response test among proven bacterial meningitis compared to others (meningism or viral meningitis [n=10 per group]).

X-axis—patient groups. Whole-blood transcript abundance was measured in duplicate in each patient via qPCR.

Y-axis overall relative abundance ratio. Nominal units. Median and Inter-quartile range exhibited.

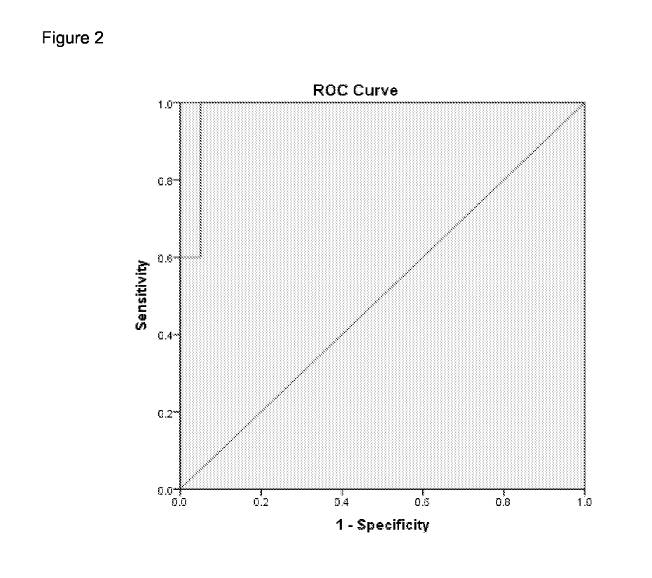

FIG. 2 is a receiver operator curve (ROC) for the 4 transcript host response test, measured via qPCR, using patients with proven bacterial meningitis (n=10) compared to others (meningism or viral meningitis [n=20]). This is based on data from FIG. 1.

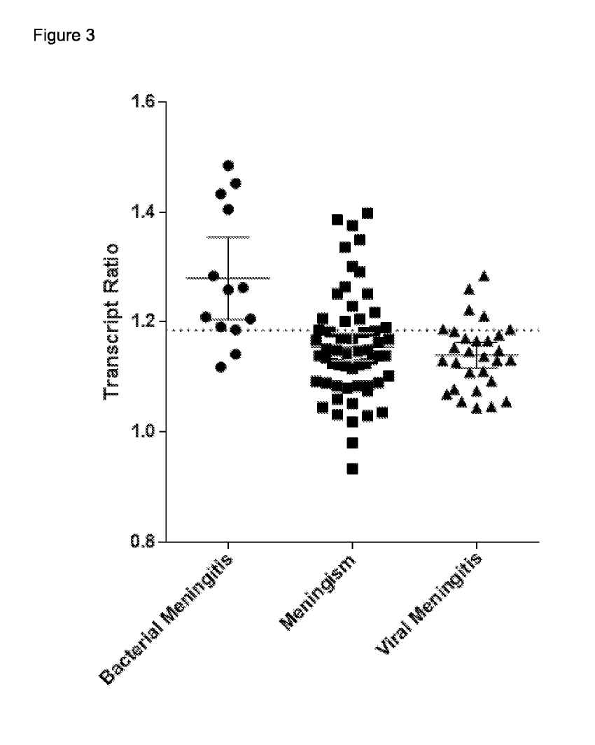

FIG. 3 is a scatter plot of overall relative abundance ratio (formulated via discriminant rule) for 4 transcript host response test among proven bacterial meningitis (n=13) compared to others (meningism or viral meningitis [n=88]).

X-axis—patient groups. Whole-blood transcript abundance was measured via qPCR in an independent sample set (n=101).

Y-axis—overall relative abundance ratio (formulated via discriminant rule). Nominal units. Median and Inter-quartile range exhibited.

Note: A low proportion of bacterial meningitis patients (13% [13/101]) was used in the sample set to reflect the prevalence of bacterial meningitis observed among adults with suspected meningitis in the UK.

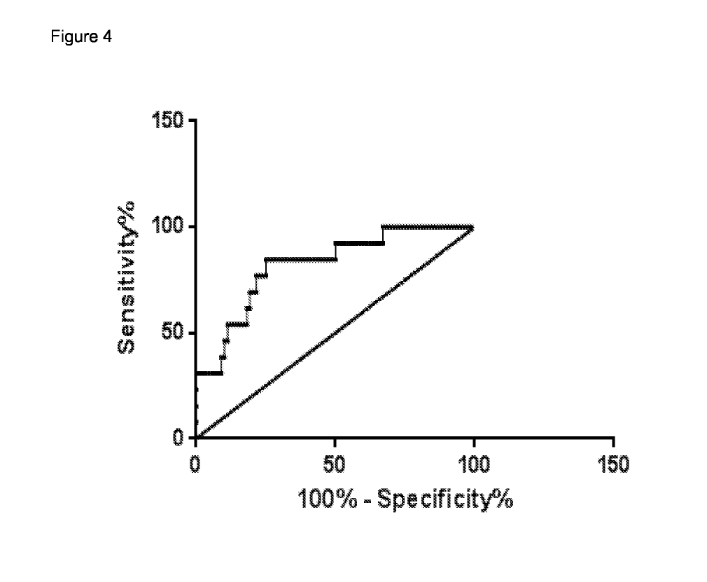

FIG. 4 is a receiver operator curve (ROC) for the 4 transcript host response test, measured via qPCR, using patients with proven bacterial meningitis (n=13) compared to others (meningism or viral meningitis [n=88]). This is based on data from FIG. 3.

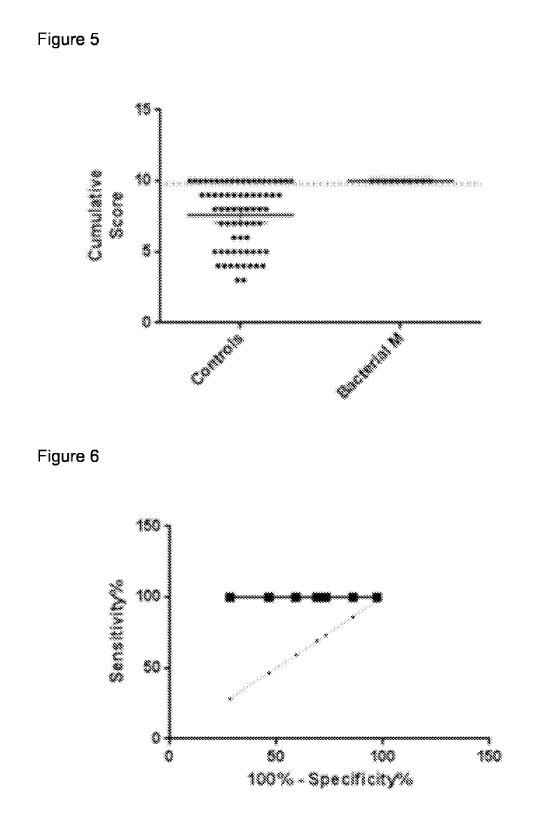

FIG. 5 is a scatter-plot of cumulative score for 12 transcript host response test. Transcript abundance measured via qPCR, using patients with proven bacterial meningitis (n=10) compared to others (meningism or viral meningitis [n=71]). Relative ratio abundances from pairs of markers (n=10) were used to provide a cumulative score.

FIG. 6 shows a receiver operator curve (ROC) for the 12 transcript host response test. Transcript abundance measured via qPCR, using patients with proven bacterial meningitis (n=10) compared to others (meningism or viral meningitis [n=71]). The ROC is based on data from FIG. 5.

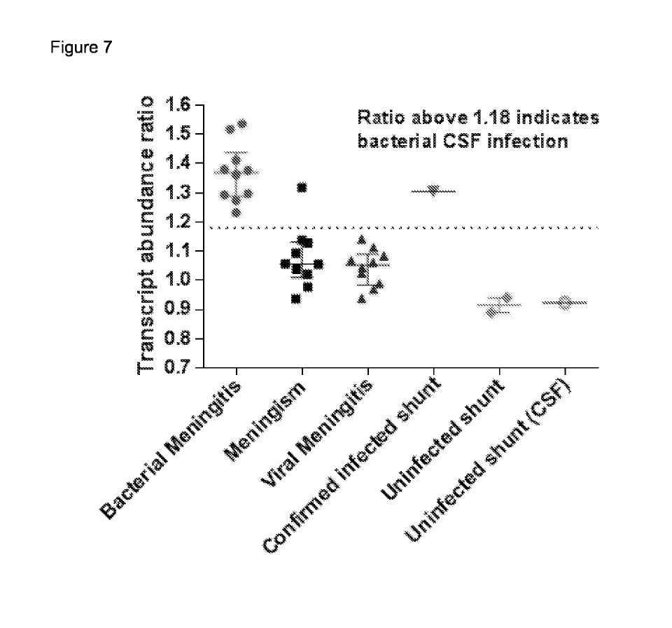

FIG. 7 is a scatter plot of relative abundance ratio (formulated via discriminant rule) for 4 transcript host response test among proven bacterial meningitis and proven ventriculo-peritoneal (VP) shunt infection. The plot shows results for proven bacterial meningitis (n=10) compared to others (meningism or viral meningitis [n=20]). In addition the plot shows results for blood from patient with confirmed ventriculo-peritoneal (VP) shunt bacterial infection (n=1); blood from patient with confirmed mechanically blocked VP shunt (n=1) with additional sample from RNA extracted from CSF of the same patient.

X-axis—patient groups. Whole-blood transcript abundance was measured via qPCR in an independent sample set (n=101).

Y-axis—overall relative abundance ratio (formulated via discriminant rule). Nominal units. Median and Inter-quartile range exhibited.

### EXPERIMENTAL RESULTS

1 Methods

1.1 Patient Selection

The blood host response assay distinguishes confirmed bacterial CSF infection (positive culture or pathogen specific PCR in CSF; or CSF pleocytosis and positive culture or pathogen specific PCR in blood) from look-a-like conditions; namely meningism or viral meningitis (patients with signs and symptoms of infection but sterile CSF; or viral specific PCR in CSF).

Discriminatory markers for proven bacterial meningitis were initially identified through whole-genome transcriptomic analysis using blood samples from suspected adult meningitis patients (n=30) recruited through UK hospitals.

These markers were then re-assessed using transcript abundance meta-data compiled from previous microarray studies among child and adult patients with proven bacterial meningitis, meningism, viral meningitis, encephalitis from UK, Nepal, India, Malawi, Vietnam populations with and without HIV co-infection (n=180). Markers that continued to distinguish bacterial meningitis from others, were then tested using a quantitative PCR (qPCR) platform re-using patient samples (n=30). Markers that remained highly discriminatory for bacterial meningitis were then validated in an independent set of UK adult patients (n=101) with suspected meningitis via qPCR.

1.2 Sample Collection

Venous whole-blood samples (2.5 mls) were collected into RNA stabilising tubes (Paxgene) during acute hospital admission (before or after antibiotic treatment had started) in consenting subjects.

1.3 RNA Extraction

Total RNA was extracted using Blood RNA Extraction kits (Paxgene) following the manufacturers' instructions.

1.4 Gene-Expression Microarrays

Total RNA (500 ng per sample) was amplified and labelled using Low Input Quick Amp. Labelling kits (Agilent). Patient sample derived amplified RNA was labelled with Cyanine-3. Control human amplified RNA (Universal RNA) was labelled with Cyanine-5. Labelled RNA (200 ng) was hybridised to SurePrint G3 GE 8×60K (Design ID 030495) human-specific microarrays following the manufacturers' instructions (Agilent Technologies). Arrays were scanned using Agilent G2505C scanner (Agilent Technologies). Raw fluorescent intensity was measured and initial quality control assessment undertaken using Agilent Feature Extraction software (FE 10.5.1.1). Transcript data were processed as previously described. Transcripts exhibiting a statistically significant difference in relative transcript abundance between proven bacterial meningitis cases and others were identified based on the fold change and false detection rate (FDR) output from SAM 4.0. (http://www-stat.stanford.edu/˜tibs/SAM).

1.5 Quantitative PCR

Complimentary DNA (cDNA) was synthesised from total RNA (500 ng per sample) using Retroscript II kit (Ambion). Quantitative PCR (qPCR) was performed with 50 ng of amplified RNA DNA using TaqMan Gene Expression Assays (Applied Biosystems) in a BioRad real-time qPCR system following the manufacturer's assay and thermo-cycler set-up instructions. Individual single-plex assays (i.e. one assay per biomarker per well) were undertaken. Relative target transcript abundance between markers was quantified using the δδCT method.

1.6 Relative Transcript Abundance Analysis

The relative transcript abundance for a set of 4 discriminatory genes (Table 2) in the microarray data was used to construct a linear discriminant function between proven bacterial (n=10) and others (n=20). The resulting discriminant rule predicted bacterial, if D>1.18, where D=1.653*log (gene A)−57.317*log(gene B)−0.577*log (gene C)+44.711*log (gene D) (FIG. 1). These genes were then validated using qPCR in the later sets of patient samples (FIGS. 3 & 4).

By way of further example, identification of the sample being as indicative of bacterial meningitis by relative transcript marker abundances can also be arrived at using different equations (which can include logical operands). An example of such an equation is as follows:

1[Bacterial Meningitis]=IF(AND(Gene A>Gene B*1.21,Gene C>Gene D*0.89,Gene B>Gene C*0.97),1,0)

D=(1.03*log(1/Gene A+GeneB)/(1/GeneC+GeneD))

Using additional discriminatory markers identified during the meta-data analysis (Table 6), the marker set was optimised using combinations of 11 additional transcripts (Table 3). In these marker sets relative ratio abundance from pairs of markers (up to 13 pairs) were used to provide a cumulative score. Further analysis of the results achieved using genes set out in Table 3 showed markers could be dropped to produce two refined marker sets without any significant drop in marker set performance (Table 4 and Table 5). All of these marker sets exhibited improved sensitivity compared to the group set out in Table 2.

2 Results

The 4 transcript host response test measured via qPCR among 30 patient samples' (samples re-used from original microarray study) exhibited 100% (10/10) sensitivity, 95% (19/20) specificity and an accuracy of 97% (29/30).

The 4 transcript host response test (Table 2) measured via qPCR among 101 patient samples' (independent sample set from microarray studies) exhibited 85% (11/13) sensitivity, 74% (65/88) specificity and an accuracy of 75% (76/101).

The 15 transcript host response test (Table 3) measured via qPCR among 81 patient samples' (independent sample set from microarray studies) exhibited 100% (10/10) sensitivity, 72% (51/71) specificity and an accuracy of 75% (61/81)

The 9 transcript host response set (Table 4) measured via qPCR among 102 patient samples' (independent sample set from microarray studies) exhibited 91% (10/11) sensitivity, 74% (68/91) specificity and an accuracy of 76% (78/102).

The 12 transcript host response test (Table 5) measured via qPCR among 81 patient samples' (independent sample set from microarray studies) exhibited 100% (10/10) sensitivity, 72% (51/71) specificity and an accuracy of 75% (61/81)—FIG. 5.

Tables of Genes

TABLE 1

| Gene | Encodes |
|---|---|
| ELANE | neutrophil elastase |
| IFI44L | Interferon-induced protein 44-like |
| CTSG | Cathepsin G |
| S1PR5 | sphingosine-1-phosphate receptor 5 |
| GRK6 | G protein-coupled receptor kinase 6 |
| TXLNA | taxilin alpha |
| DUSP1 | dual specificity phosphatase 1 |
| APMAP | adipocyte plasma membrane associated protein |
| (formerly C20orf3) | |
| KMT2A | lysine (K)-specific methyltransferase 2A |
| HP1BP3 | heterochromatin protein 1, binding protein 3 |
| SIGLEC1 | sialic acid binding Ig-like lectin 1, sialoadhesin |
| IFI27 | interferon, alpha-inducible protein 27 |
| NMT1 | N-myristoyltransferase 1 |
| FLOT1 | flotillin 1 |
| HUWE1 | HECT, UBA and WWE domain containing 1, E3 |
| | ubiquitin protein ligase |

TABLE 2

| First gene | Second gene |
|---|---|
| ELANE | IFI44L |
| CTSG | IFI44L |
| ELANE | S1PR5 |
| CTSG | S1PR5 |

TABLE 3

| First gene | Second gene |
|---|---|
| ELANE | IFI44L |
| CTSG | IFI44L |
| ELANE | S1PR5 |
| CTSG | S1PR5 |
| GRK6 | TXLNA |
| DUSP1 | IFI44L |
| APMAP | KMT2A |
| DUSP1 | HP1BP3 |
| FLOT1 | NMT1 |
| APMAP | HUWE1 |
| ELANE | SIGLEC1 |
| ELANE | IFI27 |
| DUSP1 | NMT1 |

TABLE 4

| First gene | Second gene |
|---|---|
| GRK6 | TXLNA |
| DUSP1 | IFI44L |
| APMAP | KMT2A |
| DUSP1 | HP1BP3 |
| APMAP | HUWE1 |
| DUSP1 | NMT1 |

TABLE 5

| First gene | Second gene |
|---|---|
| ELANE | IFI44L |
| CTSG | IFI44L |
| ELANE | S1PR5 |
| CTSG | S1PR5 |
| GRK6 | TXLNA |
| DUSP1 | IFI44L |
| APMAP | KMT2A |
| DUSP1 | HP1BP3 |
| ELANE | SIGLEC1 |
| ELANE | IFI27 |

TABLE 6

| | | | | |
|---|---|---|---|---|
| GRK6 | TXLNA | DUSP1 | APMAP | KMT2A |
| HP1BP3 | FLOT1 | NMT1 | HUWE1 | SIGLEC1 |
| IFI27 | ITGA4 | METTL9 | DHRS7 | LRPAP1 |
| DEFA8P | LIPN | HP | HLA-DPA1 | JUNB |
| SF1 | IRS2 | TUBA4A | IFIT2 | ATM |
| MEF2A | IMPA2 | BIN1 | PLP2 | MADD |
| TRIM26 | CNPY3 | RAF1 | RHOG | PRKCD |

1. A method of detecting a bacterial infection in a subject, the method comprising:
assaying a sample obtained from a subject to determine a relative expression level of at least one gene pair selected from the group consisting of: ELANE and IF144L; CTSG and IFI44L; ELANE and S1 PR5; and CTSG and S1 PR5; and
determining presence of a bacterial infection in the subject based on the relative expression level.

2. A method according to claim 1 comprising assaying the sample to determine the relative expression level of two gene pairs selected from the group consisting of: ELANE and IF144L; CTSG and IFI44L; ELANE and S1 PR5; and CTSG and S1 PR5.

3. A method according to claim 2 comprising assaying the sample to determine the relative expression level of three gene pairs selected from the group consisting of: ELANE and IF144L; CTSG and IFI44L; ELANE and S1 PR5; and CTSG and S1 PR5.

4. A method according to claim 2 comprising assaying the sample to determine the relative expression level of each of the gene pairs in the group consisting of: ELANE and IF144L; CTSG and IFI44L; ELANE and S1 PR5; and CTSG and S1 PR5.

5. A method of detecting a bacterial infection in a subject, the method comprising:
assaying a sample obtained from a subject to determine a relative expression level of at least one gene pair selected from the group consisting of: ELANE and IFI44L; CTSG and IFI44L; ELANE and S1 PR5; CTSG and S1 PR5; GRK6 and TXLNA; DUSP1 and IFI44L; APMAP and KMT2A; DUSP1 and HP1 BP3; FLOT1 and NMT1; APMAP and HUWE1; ELANE and SIGLEC1; ELANE and IFI27; and DUSP1 and NMT1; and
determining presence of a bacterial infection in the subject based on the relative expression level.

6. A method according to claim 5 comprising assaying the sample to determine the relative expression level of two gene pairs selected from the group consisting of: GRK6 and TXLNA; DUSP1 and IFI44L; APMAP and KMT2A; DUSP1 and HP1 BP3; APMAP and HUWE1; or DUSP1 and NMT1.

7. A method according to claim 6 comprising assaying the sample to determine the relative expression level of three gene pairs selected from the group consisting of: GRK6 and TXLNA; DUSP1 and IFI44L; APMAP and KMT2A; DUSP1 and HP1 BP3; APMAP and HUWE1; or DUSP1 and NMT1.

8. A method according to claim 7 comprising assaying the sample to determine the relative expression level of four gene pairs selected from the group consisting of: GRK6 and TXLNA; DUSP1 and IFI44L; APMAP and KMT2A; DUSP1 and HP1 BP3; APMAP and HUWE1; or DUSP1 and NMT1.

9. A method according to claim 8 comprising assaying the sample to determine the relative expression level of five gene pairs selected from the group consisting of: GRK6 and TXLNA; DUSP1 and IFI44L; APMAP and KMT2A; DUSP1 and HP1 BP3; APMAP and HUWE1; or DUSP1 and NMT1.

10. A method according to claim 9 comprising assaying the sample to determine the relative expression level of each of the gene pairs in the group consisting of: GRK6 and TXLNA; DUSP1 and IFI44L; APMAP and KMT2A; DUSP1 and HP1 BP3; APMAP and HUWE1; or DUSP1 and NMT1.

11. A method according to claim 1, further comprising conducting a confirmatory test for the bacterial infection in the subject.

12. A method according to claim 1, further comprising providing the subject with treatment for the bacterial infection.

13. A method according to claim 12, wherein the treatment comprises a therapeutically effective amount of an antibacterial agent.

14. A method according to claim 1, wherein the bacterial infection comprises bacterial meningitis caused by bacteria selected from the group consisting of: Streptococcus pneumoniae, Neisseria meningitides, Neisseria meningitides, Haemophilus influenza; and Listeria monocytogenes.

15. A method according to claim 1, wherein the sample is selected from the group consisting of: a blood sample; a cerebrospinal fluid (CSF) sample; and a synovial fluid sample.

16. (canceled)

17. A method according to claim 1, wherein assaying the sample to determine the relative expression level of at least one gene pair comprises determining relative abundance of RNA transcripts of the at least one gene pair.

18. A method according to claim 17, wherein the assay is selected from the group consisting of: RT-PCR, real-time PCR, Northern blot, RNA sequencing (RNA-seq) and RNA microarray.

19. A method according to claim 1, wherein assaying the sample to determine the relative expression level of at least one gene pair comprises determining relative abundance of proteins encoded by the at least one gene pair.

20. A method according to claim 19, wherein the assay is selected from the group consisting of: ELISA, radioimmunoassay, immunoprecipitation, Western blot and mass spectrometry.

21. (canceled)

22. A method according to claim 1, wherein the subject lacks some or all symptoms consistent with bacterial meningitis.

23.-30. (canceled)
